SEQUENCE LISTING (18 sequences)

SEQ ID NO: 1 - DNA, 23 bp, Artificial sequence
  Enterococcus faecium
tgcaaaatgctttagcaacagcc

SEQ ID NO: 2 - DNA, 23 bp, Artificial sequence
  Enterococcus faecium
tcgtgtaagctaacttcgcgtac

SEQ ID NO: 3 - DNA, 23 bp, Artificial sequence
  Enterococcus faecalis
gggtgtcttagctagcgttaacg

SEQ ID NO: 4 - DNA, 22 bp, Artificial sequence
  VanA
cgtattcttgcgcttgatgagc

SEQ ID NO: 5 - DNA, 19 bp, Artificial sequence
  VanA
cgcggacgaattggactac

SEQ ID NO: 6 - DNA, 22 bp, Artificial sequence
  VanA
gggcagagtattgacttcgttc

SEQ ID NO: 7 - DNA, 22 bp, Artificial sequence
  VanB
ggaagctatgcaagaagccatg

SEQ ID NO: 8 - DNA, 21 bp, Artificial sequence
  VanB
gggaaagccacatcaatacgc

SEQ ID NO: 9 - DNA, 23 bp, Artificial sequence
  VanC1
gctccaatctgcattaacgactg

SEQ ID NO: 10 - DNA, 23 bp, Artificial sequence
  VanC1
gccaatttcaatacccgctatcg

SEQ ID NO: 11 - DNA, 21 bp, Artificial sequence
  VanC2/C3
cgcaatcgaagcactccaatc

SEQ ID NO: 12 - DNA, 24 bp, Artificial sequence
  VanC2/C3
aaagccgtctactaatgaaatggc

SEQ ID NO: 13 - DNA, 1113 bp, Enterococcus faecium
ttgaagattactttactatatggcggacgcagcgcagagcatgaagtgtccattctttccgcattttcagttttaaatgccatttattataattattaccaagttcaactcatatttattacaaaagaaggacaatgggtcaaaggtccattactaacagaaaaacctgcaagcaaagatgtcttgcatctttcatgggacccaagtggacagacggaggaaggctttacaggaaaagtgatcaatccgggcgaaatcaaagaagaaggagccatcgtttttccagttttacatgggccaaacggggaagatggaacgatccaaggcttcttagagacattgaatatgccttatgtcggcgcaggcgtattgaccagtgcatgtgccatggataaaatcatgaccaagtatattttacaagctgctggtgtgccgcaagttccttatgtaccagtacttaagaatcaatggaaagaaaatcctaaaaaagtatttgatcaatgtgaaggttctttgctttatccgatgtttgtcaaaccggcgaatatgggttctagtgtcggcattacaaaagcagaaaaccgagaagagctgcaaaatgctttagcaacagcctatcagtatgattctcgagcaatcgttgaacaaggaattgaagcgcgcgaaatcgaagttgctgtattaggaaatgaagacgttcggacgactttgcctggtgaagtcgtaaaagacgtagcattctatgattatgaagcaaaatatatcaataataaaatcgaaatgcagattccagccgaagtgccagaagaagtttatcaaaaagcacaagagtacgcgaagttagcttacacgatgttaggcggaagcggattgagccggtgtgatttctttttgacaaataaaaatgaattattcctgaatgaattaaattctatgccaggatttacggagttcagtatgtaccccactcttatgggaaaatatgggcttgaaatacggtgatttgattgaagaactgattcaattaggaatgaatcgataccatcagcgtcaatctttttttgaaaaaaatgaataaagagaaataaagaagaagctggagtgattgcgtaa

SEQ ID NO: 14 - DNA, 1079 bp, Enterococcus faecalis
ttatttaaaacgattcaaagctaactgaatcagttcctcaattaaatcactgtatttcaagcccatattttcccacagtaaaggatacatactaaagtcagtaaaaccaggcatggtgttcaattcattcaggaataattcgtttttgcttgttaagaagaaatcacagcgacttaagccacttccatctaacataatatacgcttttttagcgtattcttgcgcttgatgagctacttcttctggaacatgcgctgggatttgcatttcaatcgtgttattgatgtattttgcatcataatcatagaaagcgacatctttcaccacttcaccaggtaaagtcgtacggacatcttcatttcctaaaatggctacttcaatttcacgtgcttcgatcccttgttcaacaattgctcgggcatcataacggaaagcttcttccaatgcttcttgcaattcttcacgattttccactttgctaattccgacactagaacccatattggcaggtttaacaaagaccggataaattaaagaaccttcacatttttcaaagacttcttttggatttcctttccagtcacttcttaaaactggcacgaatggtacttgtggaatgccaacagtttgtaaaagatatttcgtcatgattttgtccattgcgttaacgctagctaagacacccgcgcctacataaggcatattaatggtttccatgaatccttgaattgttccatcttcaccatttggcccatgtaaaacagggaaaacaatcgcttcttcttcataaatttccgaaggactgattcgttttcctgaaaattcgcctgtttcttcaggtgtttgtgcccaagttaaatgtaaaacttctttattttgtggtcgttcagataaaagagggccttttacccattgaccgtctttactaataaagactaactgtacttgataatatttataatagattgcatttaaaacggaatatgcagacaaaacagacacatcgtgctcttcacttctgccgccatacaacaaaataatcttcaaatgcttgtcctcctagtttctttcctgtcttt

SEQ ID NO: 15 - DNA, 1032 bp, Enterococcus faecium
  misc_feature: vanA
atgaatagaataaaagttgcaatactgtttgggggttgctcagaggagcatgacgtatcggtaaaatctgcaatagagatagccgctaacattaataaagaaaaatacgagccgttatacattggaattacgaaatctggtgtatggaaaatgtgcgaaaaaccttgcgcggaatgggaaaacgacaattgctattcagctgtactctcgccggataaaaaaatgcacggattacttgttaaaaagaaccatgaatatgaaatcaaccatgttgatgtagcattttcagctttgcatggcaagtcaggtgaagatggatccatacaaggtctgtttgaattgtccggtatcccttttgtaggctgcgatattcaaagctcagcaatttgtatggacaaatcgttgacatacatcgttgcgaaaaatgctgggatagctactcccgccttttgggttattaataaagatgataggccggtggcagctacgtttacctatcctgtttttgttaagccggcgcgttcaggctcatccttcggtgtgaaaaaagtcaatagcgcggacgaattggactacgcaattgaatcggcaagacaatatgacagcaaaatcttaattgagcaggctgtttcgggctgtgaggtcggttgtgcggtattgggaaacagtgccgcgttagttgttggcgaggtggaccaaatcaggctgcagtacggaatctttcgtattcatcaggaagtcgagccggaaaaaggctctgaaaacgcagttataaccgttcccgcagacctttcagcagaggagcgaggacggatacaggaaacggcaaaaaaaatatataaagcgctcggctgtagaggtctagcccgtgtggatatgtttttacaagataacggccgcattgtactgaacgaagtcaatactctgcccggtttcacgtcatacagtcgttatccccgtatgatggccgctgcaggtattgcacttcccgaactgattgaccgcttgatcgtattagcgttaaaggggtga

SEQ ID NO: 16 - DNA, 1029 bp, Enterococcus faecium
  misc_feature: vanB
atgaatagaataaaagtcgcaatcatcttcggcggttgctcggaggaacatgatgtgtcggtaaaatccgcaatagaaattgctgcgaacattgatacggaaaaattcgatccgcactacatcggaattacaaaaaacggtgtatggaagctatgcaagaagccatgtacggaatgggaagccgacagtctccccgccatactctccccggataggaaaacgcatgggctgcttgtcatgaaagaaagcgaatacgaaacacggcgtattgatgtggctttcccggttttgcatggcaaatgcggggaggatggtgcgatacaggggctgtttgtattgtctggtatcccctatgtgggctgtgatattcaaagctccgcagcttgcatggacaaatcactggcctacattcttacaaaaaatgcgggcatcgccgttcccgaatttcaaattattgataaaggtgacaagccggaggcgggtgcgcttacctaccctgtctttgtgaagccggcacggtcaggttcgtcctttggcgtaaccaaagtaaacggtacggaagaacttaacgctgcgatagaagcggcaggacaatatgatggaaaaatcttaattgagcaagcgatttcgggctgtgaggtcgggtgtgcggtcatggggaacgaggatgatttgattgtcggcgaagtggatcaaatccggctgagccacggtatcttccgcatccatcaggaaaacgagccggaaaaaggctcagaaaatgcgatgattacagttcccgcagacattccggtcgaggaacgaaatcgggtgcaggaaacggcaaagaaagtatatcgggtgcttggatgcagagggcttgcccgtgttgatctttttttgcaggaggatggcggcatcgttctaaatgaggtcaataccatgcccggttttacatcgtacagccgctacccacgtatggtggccgccgcaggaatcacgcttcctgcactgattgacagcctgattacattggcgttaaagaggtga

SEQ ID NO: 17 - DNA, 446 bp, Enterococcus gallinarum
  misc_feature: vanC1
gtcgacatgaactgccttatgttggttgccatgtcgctgcctccgcattatgtatgaacaaatggctcttgcatcaacttgctgataccatgggaatcgctagtgctcccactttgcttttatcccgctatgaaaacgatcctgccacaatcgatcgttttattcaagaccatggattcccgatctttatcaagccgaatgaagccggttcttcaaaagggatcacaaaagtaactgacaaaacagcgctccaatctgcattaacgactgcttttgcttacggttctactgtgttgatccaaaaggcgatagcgggtattgaaattggctgcggcatcttaggaaatgagcaattgacgattggtgcttgtgatgcgatttctcttgtcgacggtttttttgattttgaagagaaataccaattaatcagcgccacgacanaaaaa

SEQ ID NO: 18 - DNA, 1086 bp, Enterococcus casseliflavus
  misc_feature: vanC2/C3
gaatcggaaaagcggaaggaagaaaaacatgaaaaaaatcgccattatttttggaggcaattcaccggaatacaccgtttctttagcttcagcaactagcgcaatcgaagcactccaatcatctccctatgactacgacctctctttgatcgggatcgccccagatgctatggattggtacttgtatacaggagaactggaaaacatccgacaagacacgtggttgttggatacgaaacataaacagaaaatacagccgctattcgaaggaaacggcttttggctaagtgaagagcagcaaacgttggtacctgatgttttatttcccattatgcatggcaaatacggggaagatggcagtatccaaggattgtttgaattgatgaagctgccttatgtaggctgcggggtggcaggttctgccttatgtatgaacaaatggctgctgcatcaagctgcagcagccattggcgtacaaagtgctcctacgattctcttgacaaatcaagccaaccagcaagaacaaatcgaagcttttatccagacccatggcttcccagttttctttaagcctaatgaagcgggctcctcaaaagggatcactaaagtcacctgcgttgaagaaatcgcttctgccttaaaagaagcctttacttattgttccgcagtgctcctacaaaaaaatattgccggtgttgagatcggttgcggtattttgggcaacgactctttgactgtcggtgcttgtgacgccatttcattagtagacggctttttcgattttgaagaaaagtaccagctgatcagcgccaaaatcaccgtccctgcgccattgcctgaaacgattgaaaccaaggtcaaagaacaagctcagctgctctatcgtagtcttggtcttaaaggtcttgctcgcatcgacttttttgtcacggagcgaggagaactatacttgaatgaaatcaatactatgccgggctttacgagtcactcccgctatcctgccatgatggcagcggtcggcttatcctatcaagaactactacaaaaactgcttgtcttagcaaaggaggaagtcaaatgaatccc

(19) United States
(12) Patent Application Publication
(10) Pub. No.: US 2012/0171681 A1
(43) Pub. Date: Jul. 5, 2012

(54) OLIGONUCLEOTIDES, METHODS AND KITS FOR DETECTING AND IDENTIFYING VANCOMYCIN-RESISTANT ENTEROCOCCUS

(76) Inventors: Rodrigo Malig, Santiago (CL); Francisco Melo, Santiago (CL); Gaelle Lehouque, Sao Paulo (BR); Denis Berndt, Santiago (CL)

(21) Appl. No.: 13/145,242

(22) PCT Filed: Jan. 19, 2009
(86) PCT No.: PCT/EP09/00299

§ 371 Date: Feb. 2, 2012

(51) Int. Cl. C12Q 1/68; C07H 21/04

(52) U.S. Cl. 435/6.12; 435/6.15; 536/23.1

(57) ABSTRACT

The present invention relates to methods for detecting the presence or absence of vancomycin resistant enterococci (VRE) in a sample using a multiplex polymerase chain reaction (mtx-PCR) assay. Furthermore, the present invention relates to oligonucleotide primers and kits for the detection of VRE.

### FIELD OF THE INVENTION

This invention relates to bacterial diagnostics by using molecular biological methods, and specifically relates to detection of enterococci in a sample by amplifying fragments of their nucleic acids and detecting the amplified nucleic acid sequences.

### BACKGROUND OF THE INVENTION

Enterococci are Gram-positive cocci that are considered normal inhabitants of the gastrointestinal tract and the female genital tract. Enterococcus spp. are not particularly pathogenic in humans, but vancomycin-resistant enterococci have been increasingly identified as an important cause of hospital acquired infection. In fact, VRE have been recognized as the second most common cause of hospital infection. At least three different phenotypes associated with the gene cassettes vanA, vanB and vanC are responsible for vancomycin resistance in enterococci. Enterococcus faecalis and Enterococcus faecium are clinically significant species that are implicated in 90% and 5-10% of enterococcal infections, respectively.

The typical reservoirs for VRE are colonized and infected patients. The Centers for Disease Control and Prevention (CDC) and the Society of Healthcare Epidemiology of America recognized the importance of active VRE surveillance to reduce or eliminate hospital-acquired infections (Centers for Disease Control and Prevention. 1995. Morb. Mortal. Wkly. Rep. Recomm. Rep. 44:1-13; Muto et al. 2003. Infect. Control Hosp. Epidemiol. 24:362-386). Any established surveillance program will benefit from the fast identification of VRE carriers by allowing the rapid isolation of those patients, thus minimizing the spread of infection.

Although bacteria culture is currently the method of choice for VRE screening, this technique has the main drawbacks of long turn-around time (48-72 hrs) (Novicki et al. 2004. J. Clin. Microbiol. 42:1637-1640; Van Horn et al. 1996. J. Clin. Microbiol. 34:2042-2044) and limited detection sensitivity (D'Agata et al. 2002. Clin. Infect. Dis. 34:167-172). Therefore, the availability of an assay capable of detecting VRE in few hours, followed by an appropriate infection control program, could reduce or eliminate hospital-acquired VRE infections, thus leading to a significant reduction of patient mortality by this particular cause.

Other types of assays have been developed into more rapid diagnostic tools such as immunoassays, including radioimmunoassays, enzyme-linked immunoassays and latex agglutination and immunoblotting assays. Additionally, PCR-based assays are rapidly gaining popularity in clinical laboratory practice. Some nucleic acid amplification tests have been developed and evaluated for the detection of VRE. Patel and colleagues developed a multiplex PCR-RFLP assay for the simultaneous detection and identification of vanA, vanB, vanC1 and vanC2/C3 genes (Patel, et al. 1997. J. Clin. Microbiol. 35: 703-707). However, this assay needs a digestion step for identifications of PCR products, which makes it difficult of implementing as a routine assay. Also, some patents have demonstrated the detection of vanA and vanB genes (WO/2005/028679, US 2005/0058985 A1, U.S. Pat. No. 7,074,598) but these assays deliver less information than the culture strategy (species identification and detection of vanC genotypes), which limits the full integration of molecular approaches for VRE detection.

Although the above methods can be used to detect VanA/VanB genotypes, there is an urgent need for a rapid and reliable method for simultaneously detecting VRE (E. faecium, E. faecalis and VanA, VanB, VanC1, VanC2/C3 genotypes), both in the hospital and community settings. The present invention provides oligonucleotide primers and methods for detecting VRE (E. faecium, E. faecalis and VanA, VanB, VanC1, VanC2/C3 genotypes) easily and rapidly from patient samples.

### SUMMARY OF THE INVENTION

The invention provides oligonucleotides, methods and kits for identifying clinically relevant enterococcal species and vancomycin-resistant genotypes in a biological sample. Oligonucleotide primers for detecting specific nucleotide targets of Enterococcus faecium and Enterococcus faecalis species, in addition to vanA, vanB, vanC1 and vanC2/C3 (genes associated with vancomycin resistance of microorganisms) genes are provided by the invention, as well as kits containing such oligonucleotide primers. Methods of the invention can be used for the rapid identification of VRE nucleic acids from samples.

In one aspect of the invention, there is provided a method for detecting the presence or absence of one or more VRE in a biological sample. The method to detect VRE consists of a multiplex PCR amplification reaction coupled to detection and identification of PCR products. The PCR amplification step includes mixing the sample with target specific primer pairs (where the targets are: E. faecium, E. faecalis, VanA, VanB, VanC1, and VanC2/C3) to produce several PCR amplification products (one per each target present in the sample). The detection and identification of PCR amplification products could be carried out by using any method well known to those of ordinary skill in the art, as for example: gel electrophoresis, capillary electrophoresis, gel microfluidics, high resolution melt, probe hybridization, etc. The detection of at least two amplification products, one belonging to an enterococcal species and the another belonging to a vancomycin-resistance genotype are typically indicative of the presence of VRE, while the absence of this condition indicates the contrary.

The E. faecium specific primer pair includes the following sequences: 5′-TGC AAA ATG CTT TAG CAA CAG CC-3′ (SEQ ID NO:1) and 5′-TCG TGT AAG CTA ACT TCG CGT AC-3′ (SEQ ID NO:2). The E. faecium specific primer pair may also comprise oligonucleotides substantially corresponding to SEQ ID NO:1 and SEQ ID NO:2, or the complement thereof, or a portion thereof.

The E. faecalis specific primer pair includes the following sequences: 5′-CGT ATT CTT GCG CTT GAT GAG C-3′ (SEQ ID NO:3) and 5′-GGG TGT CTT AGC TAG CGT TAA CG-3′ (SEQ ID NO:4). The E. faecalis specific primer pair may also comprise oligonucleotides substantially corresponding to SEQ ID NO:3 and SEQ ID NO:4, or the complement thereof, or a portion thereof.

The VanA specific primer pair includes the following sequences: 5′-CGC GGA CGA ATT GGA CTA C-3′ (SEQ ID NO:5) and 5′-GGG CAG AGT ATT GAC TTC GTT C-3′ (SEQ ID NO:6). The VanA specific primer pair may also comprise oligonucleotides substantially corresponding to SEQ ID NO:5 and SEQ ID NO:6, or the complement thereof, or a portion thereof.

The VanB specific primer pair includes the following sequences: 5′-GGA AGC TAT GCA AGA AGC CAT G-3′ (SEQ ID NO:7) and 5′-GGG AAA GCC ACA TCA ATA CGC-3′ (SEQ ID NO:8). The VanB specific primer pair may also comprise oligonucleotides substantially corresponding to SEQ ID NO:7 and SEQ ID NO:8, or the complement thereof, or a portion thereof.

The VanC1 specific primer pair includes the following sequences: 5′-GCT CCA ATC TGC ATT AAC GAC TG-3′ (SEQ ID NO:9) and 5′-GCT CCA ATC TGC ATT AAC GAC TG-3′ (SEQ ID NO:10). The VanC1 specific primes pair may also comprise oligonucleotides substantially corresponding to SEQ ID NO:9 and SEQ ID NO:10, or the complement thereof, or a portion thereof.

The VanC2/C3 specific primer pair includes the following sequences: 5′-CGC AAT CGA AGC ACT CCA ATC-3′ (SEQ ID NO:11) and 5′-AAA GCC GTC TAC TAA TGA AAT GGC-3′ (SEQ ID NO:12). The VanC2/C3 specific primer pair may also comprise oligonucleotides substantially corresponding to SEQ ID NO:11 and SEQ ID NO:12, or the complement thereof, or a portion thereof.

The invention includes a method to detect the presence of VRE in a biological sample. Representative biological samples include anal or perirectal swabs, stool samples, blood and body fluids. In one embodiment, the sample is from a culture (eg. a portion of, or an individual colony including those from an enriched culture, or from a liquid culture). The method includes providing, one or more oligonucleotide primers and subjecting an amplification reaction at conditions effective to amplify any of the target sequences (E. faecium, E. faecalis, VanA, VanB, VanC1, and VanC2/C3). In one embodiment, the amplification reaction mixture includes all oligonucleotide primers in a single mix, and subjecting the amplification reaction mixture to conditions effective to amplify all target sequences simultaneously in a single reaction.

In another embodiment, the amplification reaction mixture includes some of all oligonucleotide primers in a single mix, and subjecting the amplification reaction mixture to conditions effective to amplify some target sequences in a single reaction. In an alternative embodiment, independent amplification reactions are conducted, one for each target. In another embodiment, a single amplification reaction is conducted for detecting one target sequence.

In another embodiment, the amplification reaction can include control oligonucleotide primers and control target DNA to monitor the presence of PCR inhibitors in the sample.

The above-described methods can further include preventing amplification of a contaminant nucleic acid. Preventing amplification of a contaminant nucleic acid could be done by using any method well known to those of ordinary skill in the art, as for example performing the PCR amplification step in the presence of uracil and treating the biological sample with uracil-DNA glycosylase prior to amplifying. In addition, the PCR amplification step can be performed in a control sample. A control sample may include any of the above-mentioned targets nucleic acid molecules.

In another aspect of the invention, there are provided articles of manufacture or kits. Kits of the invention include the following E. faecium specific primer pair 5′-TGC AAA ATG CTT TAG CAA CAG CC-3′ (SEQ ID NO:1) and 5′-TCG TGT AAG CTA ACT TCG CGT AC-3′ (SEQ ID NO:2), and also may include oligonucleotides substantially corresponding to SEQ ID NO:1 and SEQ ID NO:2, or the complement thereof, or a portion thereof.

Articles of manufacture of the invention can further or alternatively include the following E. faecalis specific primer pair 5′-CGT ATT CTT GCG CTT GAT GAG C-3′ (SEQ ID NO:3) and 5′-GGG TGT CTT AGC TAG CGT TAA CG-3′ (SEQ ID NO:4), and may also include oligonucleotides substantially corresponding to SEQ ID NO:3 and SEQ ID NO:4, or the complement thereof, or a portion thereof.

Articles of manufacture of the invention can further or alternatively include the following VanA specific primer pair 5′-CGC GGA CGA ATT GGA CTA C-3′ (SEQ ID NO:5) and 5′-GGG CAG AGT ATT GAC TTC GTT C-3′ (SEQ ID NO:6), and may also include oligonucleotides substantially corresponding to SEQ ID NO:5 and SEQ ID NO:6, or the complement thereof, or a portion thereof.

Articles of manufacture of the invention can further or alternatively include the following VanB specific primer pair 5′-GGA AGC TAT GCA AGA AGC CAT G-3′ (SEQ ID NO:7) and 5′-GGG AAA GCC ACA TCA ATA CGC-3′ (SEQ ID NO:8), and may also include oligonucleotides substantially corresponding to SEQ ID NO:7 and SEQ ID NO:8, or the complement thereof, or a portion thereof.

Articles of manufacture of the invention can further or alternatively include the following VanC1 specific primer pair. 5′-GCT CCA ATC TGC ATT AAC GAC TG-3′ (SEQ ID NO:9) and 5′-GCT CCA ATC TGC ATT AAC GAC TG-3′ (SEQ ID NO:10), and may also include oligonucleotides substantially corresponding to SEQ ID NO:9 and SEQ ID NO:10, or the complement thereof, or a portion thereof.

Articles of manufacture of the invention can further or alternatively include the following VanC2/C3 specific primer pair: 5′-CGC AAT CGA AGC ACT CCA ATC-3′ (SEQ ID NO:11) and 5′-AAA GCC GTC TAC TAA TGA AAT GGC-3′ (SEQ ID NO:12), and may also include oligonucleotides substantially corresponding to SEQ ID NO:11 and SEQ ID NO:12, or the complement thereof, or a portion thereof.

In one embodiment, the oligonucleotides of the invention include sequences substantially corresponding to nucleotides 593 to 615 of the ddl gene from E. faecium (SEQ ID NO:1; an exemplary of Ddl gene from E. faecium has SEQ ID NO:13), or the complement thereof, or a portion thereof; sequences substantially corresponding to nucleotides 831 to 853 of the ddl gene from E. faecium (reverse-complemented of SEQ ID NO:2; an exemplary of ddl gene from E. faecium has SEQ ID NO:13), or the complement thereof, or a portion thereof, sequences substantially corresponding to nucleotides 213 to 234 of the Ddl gene from E. faecalis (SEQ ID NO:3; an exemplary of ddl gene from E. faecalis has SEQ ID NO:14), or the complement thereof, or a portion thereof; sequences substantially corresponding to nucleotides 663 to 685 of the Ddl gene from E. faecalis (reverse-complemented of SEQ ID NO:4; an exemplary of Ddl gene from E. faecalis has SEQ ID NO:14), or the complement thereof, or a portion thereof; sequences substantially corresponding to nucleotides 558 to 576 of the vanA gene (SEQ ID NO:5; an exemplary of vanA gene has SEQ ID NO:15), or the complement thereof, or a portion thereof; sequences substantially corresponding to nucleotides 909 to 930 of the vanA gene (reverse-complemented of SEQ ID NO:6; ₐₙ exemplary of vanA gene has SEQ ID NO:15), or the complement thereof, or a portion thereof; sequences substantially corresponding to nucleotides 146 to 167 of the vanB gene (SEQ ID NO:7; an exemplary of vanB gene has SEQ ID NO:16), or the complement thereof, or a portion thereof; sequences substantially corresponding to nucleotides 264 to 284 of the vanB gene (reverse-complemented of SEQ ID NO:8; an exemplary of vanB gene has SEQ ID NO:16), or the complement thereof, or a portion thereof; sequences substantially corresponding to nucleotides 248 to 270 of the vanC1 gene (SEQ ID NO:9; an exemplary of vanC1 gene has SEQ ID NO:17), or the complement thereof, or a portion thereof; sequences substantially corresponding to nucleotides 307 to 329 of the vanC1 gene (reverse-complemented of SEQ ID NO:10; an exemplary of vanC1 gene has SEQ ID NO:17), or the complement thereof, or a portion thereof; sequences substantially corresponding to nucleotides 100 to 120 of the vanC2/C3 gene (SEQ ID NO:11; an exemplary of vanC2/C3 gene has SEQ ID NO:18), or the complement thereof, or a portion thereof; sequences substantially corresponding to nucleotides 752 to 775 of the vanC2/C3 gene (reverse-complemented of SEQ ID NO:12; an exemplary of vanC2/C3 gene has SEQ ID NO:18), or the complement thereof, or a portion thereof.

The invention further includes a kit with primers useful to amplify ddl gene from E. faecium and/or ddl gene from E. faecalis and/or the vanA gene and/or the vanB gene and/or VanC1 gene and/or VanC2/C3 gene in a test sample. The kit includes one or more oligonucleotide comprising sequences corresponding to nucleotides 593 to 615 of the ddl gene from E. faecium (SEQ ID NO:1; an exemplary of Ddl gene from E. faecium has SEQ ID NO:13), or the complement thereof, or a portion thereof; sequences substantially corresponding to nucleotides 831 to 853 of the ddl gene from E. faecium (reverse-complemented of SEQ ID NO:2; an exemplary of ddl gene from E. faecium has SEQ ID NO:13), or the complement thereof, or a portion thereof; sequences substantially corresponding to nucleotides 213 to 234 of the Ddl gene from E. faecium (SEQ ID NO:3; an exemplary of ddl gene from E. faecalis has SEQ ID NO:14), or the complement thereof, or a portion thereof; sequences substantially corresponding to nucleotides 663 to 685 of the Ddl gene from E. faecium (reverse-complemented of SEQ ID NO:4; an exemplary of Ddl gene from E. faecalis has SEQ ID NO:14), or the complement thereof, or a portion thereof; sequences substantially corresponding to nucleotides 558 to 576 of the vanA gene (SEQ ID NO:5; an exemplary of vanA gene has SEQ ID NO:15), or the complement thereof, or a portion thereof; sequences substantially corresponding to nucleotides 909 to 930 of the vanA gene (reverse-complemented of SEQ ID NO:6; an exemplary of vanA gene has SEQ ID NO:15), or the complement thereof, or a portion thereof; sequences substantially corresponding to nucleotides 146 to 167 of the vanB gene (SEQ ID NO:7; an exemplary of vanB gene has SEQ ID NO:16), or the complement thereof, or a portion thereof; sequences substantially corresponding to nucleotides 264 to 284 of the vanB gene (reverse-complemented of SEQ ID NO:8; an exemplary of vanB gene has SEQ ID NO:16), or the complement thereof, or a portion thereof; sequences substantially corresponding to nucleotides 248 to 270 of the vanC1 gene (SEQ ID NO:9; an exemplary of vanC1 gene has SEQ ID NO:17), or the complement thereof, or a portion thereof; sequences substantially corresponding to nucleotides 307 to 329 of the vanC1 gene (reverse-complemented of SEQ ID NO:10; an exemplary of vanC1 gene has SEQ ID NO:17), or the complement thereof, or a portion thereof; sequences substantially corresponding to nucleotides 100 to 120 of the vanC2/C3 gene (SEQ ID NO:11; an exemplary of vanC2/C3 gene has SEQ ID NO:18), or the complement thereof, or a portion thereof; sequences substantially corresponding to nucleotides 752 to 775 of the vanC2/C3 gene (reverse-complemented of SEQ ID NO:12; an exemplary of vanC2/C3 gene has SEQ ID NO:18), or the complement thereof, or a portion thereof. The kit may optionally include other oligonucleotide primers useful to amplify or detect other genes, including other drug resistance genes.

Articles of the invention can include labelled oligonucleotide primers. The article of manufacture can also include a package label or package insert having instructions there for using the oligonucleotide primer pairs falling under the scope of this invention to detect the presence or absence of one or more VRE in a biological sample.

Unless otherwise defined, all technical and scientific terms used herein have the same meaning as commonly understood by one of ordinary skill in the art to which this invention belongs. Although methods and materials similar or equivalent to those described herein can be used in the practice or for testing of the present invention, suitable methods and materials are described below. In addition, the materials, methods and examples are illustrative only, and thus not intended to be limiting.

### DETAILED DESCRIPTION

Definitions

As used herein, the following terms have the following definitions:

A “nucleotide” is a subunit of a nucleic acid comprising a purine or pyrimidine base group, a 5-carbon sugar and a phosphate group. The 5-carbon sugar found in RNA is ribose. In DNA, the 5-carbon sugar is 2′-deoxyribose. The term also includes analogs of such subunits, such as a methoxy group (MeO) at the 2′position of ribose.

An “oligonucleotide” is a polynucleotide having two or more nucleotide subunits covalently joined together. Oligonucleotides are generally about 10 to about 100 nucleotides in length, or more preferably 8 to 50 nucleotides in length. The sugar groups of the nucleotide subunits may be ribose, deoxyribose, or modified derivatives thereof. The nucleotide subunits may be joined by linkages such as phosphodiester bonds, modified linkages or by non-nucleotide moieties that do not prevent hybridization of the oligonucleotide to its complementary target nucleotide sequence. Modified linkages include those in which a standard phosphodiester bond is replaced with a different linkage, such as a phosphorothioate linkage, a methylphosphonate linkage, or a neutral peptide linkage. Nitrogenous base analogs may also be components of oligonucleotides in accordance with the invention. Ordinarily, oligonucleotides will be synthesized by organic chemical methods and will be single-stranded unless specified otherwise. Oligonucleotides can be labelled with a detectable chemical group. Any oligonucleotide sequences optimally designed to be used by the method described in this invention may be prepared by any known procedure, including synthetic, recombinant, ex vivo generation, or a combination thereof, as well as utilizing any purification methods known in the art.

A “target” is a nucleic acid from E. faecalis, E. faecium or from VanA, VanB, VanC1 or VanC2/C3 genes.

A “target nucleic acid sequence”, “target nucleotide sequence” or “target sequence” is a specific deoxyribonucleotide or ribonucleotide that can be hybridized by a “target specific primer”.

A “target specific primer” or “primer” is a single-stranded oligonucleotide that combines with a complementary single-stranded target to form a double-stranded hybrid. A “target specific primer pair” is two target specific primers that in contact with target sequences may generate a PCR amplification product from that target.

“E. faecium specific primer pair” as used herein refers to oligonucleotide primers that anneal specifically to portions of E. faecium genome, and initiate synthesis therefrom under appropriate conditions.

“E. faecalis specific primer pair” as used herein refers to oligonucleotide primers that anneal specifically to portions of E. faecalis genome, and initiate synthesis therefrom under appropriate conditions.

“VanA specific primer pair” as used herein refers to oligonucleotide primers that anneal specifically to vanA nucleic acid sequences, and initiate synthesis therefrom under appropriate conditions.

“VanB specific primer pair” as used herein refers to oligonucleotide primers that anneal specifically to vanB nucleic acid sequences, and initiate synthesis therefrom under appropriate conditions.

“VanC1 specific primer pair” as used herein refers to oligonucleotide primers that anneal specifically to vanC1 nucleic acid sequences, and initiate synthesis therefrom under appropriate conditions.

“VanC2/C3 specific primer pair” as used herein refers to oligonucleotide primers that anneal specifically to vanC2/C3 nucleic acid sequences, and initiate synthesis therefrom under appropriate conditions.

A “biological sample” refers to a sample of material that is to be tested for the presence of microorganisms or nucleic acid thereof. The biological sample can be obtained from an organism (eg. it can be a physiological sample, such as one from a human patient, for example, blood sample, sputum sample, spinal fluid sample, a urine sample, a stool sample, a rectal swab, a peri-rectal swab, a nasal swab, a throat swab, or a culture of such a sample), a colony on a plate or a liquid culture. Ordinarily, the biological sample will contain polynucleotides. These polynucleotides may have been released from organisms that comprise the biological sample, or alternatively can be released from the organisms in the sample using techniques such as sonic disruption or enzymatic or chemical lysis of cells to release polynucleotides so that they are available for amplification with one or more primers.

“Melting temperature” or “Tm” refers to the temperature at which 50% of the probe or primer is present in its hybridized form with the target sequence (or alternatively, the temperature at which 50% of the probe or primer is found free in solution, not interacting with its target sequence).

One skilled in the art will understand that primers that substantially correspond to a reference sequence or region can vary from that reference sequence or region and still hybridize to the same target nucleic acid sequence. Primers of the present invention substantially correspond to a nucleic acid sequence or region if the percentage of identical bases or the percentage of perfectly complementary bases between the primer and its target sequence is from 100% to 60% or from 0 base mismatches in a 10 nucleotide target sequence to 4 bases mismatched in a 10 nucleotide target sequence. In one embodiment, the percentage is from 80% to 100%. In another embodiment this percentage is from 90% to 100%; and in yet other embodiments, this percentage is from 95% to 100%. Primers that substantially correspond to a reference sequence or region include primers having any additions or deletions which do not prevent the primer from having its claimed property, such as being able to preferentially hybridize to its target nucleic acid over non-target nucleic acids.

The various components of the present invention are described in greater detail below. It should be appreciated that while certain embodiments are discussed in regard to these components, other methods known in the art for accomplishing the same ends should be considered within the scope of the present disclosure. In addition, various embodiments of the present invention may use different methods for carrying out the steps described below, depending on the purpose of the method.

Overview

The present invention provides methods for detecting the presence or absence of VRE in a biological sample. Primers for detecting E. faecalis, E. faecium and/or vanA, vanB, vanC1 and/or vanC2/C3 nucleic acids are provided by the invention, as are articles of manufacture containing such primers. The assay was designed to detect all VRE targets simultaneously in a single reaction. The increased sensitivity of VRE detection and speed of the PCR assay, as compared to other methods, make feasible the implementation of this technology for routine clinical laboratory for the detection of VRE.

The total time for processing a sample using the VRE assay provided in this invention is less than 4 hrs, as compared to the 4-7 days required for detection by routine culture. The invention has the potential to replace standard culture methods, which require selective media, biochemical testing and susceptibility testing, thus resulting in cost savings to institutions. Using the method provided in this invention, clinicians receive a single test result within a few hours, thus allowing that appropriate isolation procedures and antimicrobial therapy can begin almost immediately. The rapid VRE assay allows hospitals to take the necessary precautions with VRE-infected patients such that the spread of VRE to other patients is prevented.

Enterococcus spp.

Enterococcus spp. have the characteristic of being resistant to many antimicrobial agents, which make them formidable pathogens and limit the therapeutic options available to the clinician. All enterococci are intrinsically resistant to a number of antibiotics and exhibit low levels of resistance to the β-lactam agents, the aminogycosides, and the lincosamides. They have acquired genes of resistance to all known antimicrobial agents, including the glycopeptides vancomycin and teicoplanin. One of the concerns is the possibility that the vancomycin-resistant genes may be transferred to other Gram-positive organisms, especially Staphylococcus aureus.

A sequence similarity search by BLAST software against current sequence databases found no other organisms containing sequences similar to vanA gene. The same procedure for vanB genes revealed that similar sequences can be found in Enterococcus spp. and animal species (veal calves) of streptococci such as S. gallolyllcus and S. infantarius (Genebank Accession Nos. AY035705 and Z70527). One other isolate, S. bovis, also has sequences that exhibit homology to vanB sequences. These Streptococcus isolates appear to have acquired enterococcal vanB vancomycin resistance genes.

VRE are able to hydrolyze esculin, exhibit optimal growth at 35° C. and 6.5% NaCl. VRE are selectively cultured on Enterococcosel agar containing 6 μg/ml vancomycin. The glycopeptide resistance of VRE has three different phenotypes. vanA is the most frequently isolated phenotype with high levels of resistance to vancomycin and teicoplanin. The vanB phenotypes (eg. vanB, or vanB-2/3) have variable vancomycin-resistance and are susceptible to teicoplanin. The vanC phenotype (eg. vanC1, or vanC2/C3) has low levels of vancomycin-resistance and is susceptible to teicoplanin. The enterococcal species identification is done on isolated colonies using biochemical methods. Alternatively, automated biochemical assays can also be used, as for example with a GPI card in a VITEK equipment (Biomerieux, France).

Teicoplanin disc susceptibility test can be used to differentiate the vanA genotype from the vanB genotype. The vanA strains typically exhibit a high level of vancomycin resistance (minimum inhibitory concentration or MIC>64 μg/ml). VanA strains also exhibit inducible resistance to vancomycin and teicoplanin. The genes encoding vanA are located on a transposon or a plasmid, and are easily transferred by conjugation. The first vanA strain of vancomycin-resistant enterococci was reported in 1986 and represents approximately 70% of vancomycin-resistant enterococci isolates from patient specimens. On the other hand, vanB strains exhibit variable resistance to vancomycin (MIC 4 to >1024 μg/ml), and exhibit inducible resistance to vancomycin only. The genes encoding vanB are chromosomal and can be transferred by conjugation. VanB strains were first identified in the U.S. in 1987 and currently make up about 25% of the vancomycin-resistant patient isolates.

Vancomycin-resistant Enterococci Nucleic Acids and Oligonucleotides

The invention provides methods to detect E. faecium, E. faecalis, vanA, vanB, vanC1, and vanC2/C3 nucleic acids by PCR amplification. Specifically, primers to amplify and detect E. faecium, E. faecalis, vanA, vanB, vanC1, and vanC2/C3 nucleic acids are provided by the invention.

The oligonucleotide primers were designed using the following nucleic acid templates:
I. E. faecalis specific primer pair was designed from E. faecalis' D-alanine:D-alanine ligase (ddl) gene.
II. E. faecium specific primer pair was designed from E. faecium's D-alanine:D-alanine ligase (ddl) gene.
III. VanA specific primer pair was designed from VanA gene.
IV. VanB specific primer pair was designed from VanB gene.
V. VanC1 specific primer pair was designed from VanC1 gene.
VI. VanC2/C3 specific primer pair was designed from VanC2/C3 gene.

The oligonucleotide primers were designed by optimizing several restraints that include, but are not limited to: avoid the occurrence of primer-primer interactions to allow the parallel amplification of all targets in the same reaction tube, generation of amplification products with different sizes to allow the simultaneous detection of several targets (eg. by electrophoresis), similar melting temperatures of primers, primer specificity (ie. avoid the hybdridization of primers with non-target nucleic acids that are likely to be present in the sample), and primer length (ie. the primers need to be long enough to anneal with sequence-specificity and to initiate synthesis but not so long that fidelity is reduced during oligonucleotide synthesis). Typically, oligonucleotide primers are 8 to 50 nucleotides in length. The optimal design of PCR primers can be achieved by the integration of ordinary bioinformatics software. Any skilled in the art can create the necessary software, or use existing software, to accomplish the optimized design of the oligonucleotides primers. The primers provided by the present invention were designed by our own custom software. The careful design of the oligonucleotide primers contributes to insure a high specificity and to maximize the multiplexing capacity.

Based on such considerations, the following regions were chosen to design oligonucleotides:
I. From the ddl gene from E. faecium nucleotides 593 to 615 and nucleotides 831 to 853 of the ddl gene having SEQ ID NO:13.
II. From the ddl gene from E. faecalis nucleotides 213 to 234 and nucleotides 663 to 685 of the ddl gene having SEQ ID NO:14.
III. From the vanA gene from nucleotides 558 to 576 and nucleotides 909 to 930 of the vanA gene having SEQ ID NO:15.
IV. From the vanB gene from nucleotides 146 to 167 and nucleotides 264 to 284 of the vanB gene having SEQ ID NO:16.
V. From the vanC1 gene from nucleotides 248 to 270 and nucleotides 307 to 329 of the vanC1 gene having SEQ ID NO:17.
VI. From the vanC2/C3 gene from nucleotides 100 to 120 and nucleotides 752 to 775 of the vanC2/C3 gene having SEQ ID NO:18.

In one embodiment, the ddl gene from E. faecium oligonucleotides include SEQ ID NO: 1 or 2, the complement or a portion thereof, the ddl gene from E. faecalis oligonucleotides include SEQ ID NO: 3 or 4, the complement or a portion thereof, the vanA gene oligonucleotides include SEQ ID NO: 5 or 6, the complement or a portion thereof, the vanB gene oligonucleotides include SEQ ID NO: 7 or 8, the complement or a portion thereof, the vanC1 gene oligonucleotides include SEQ ID NO: 9 or 10, the complement or a portion thereof, and the vanC2/C3 gene oligonucleotides include SEQ ID NO: 11 or 12, the complement or a portion thereof.

Once presumptive sequences have been identified, corresponding oligonucleotides are produced. Defined oligonucleotides that can be used to practice the invention can be produced by any of several well-known methods, including automated solid-phase chemical synthesis using phosphoramidite precursors. Other well-known methods for construction of synthetic oligonucleotides may, of course, be employed. All of the oligonucleotides of the present invention may be modified with chemical groups to enhance their performance. Backbone-modified oligonucleotides, such as those having phosphorothioate or methylphosphonate groups, are examples of analogs that can be used in conjunction with oligonucleotides of the present invention. These modifications render the oligonucleotides resistant to the nucleolytic activity of certain polymerases or to nuclease enzymes. Other analogs that can be incorporated into the structures of the oligonucleotides include peptide nucleic acids, or “PNAs”. The PNAs are compounds comprising ligands linked to a peptide backbone rather than to a phosphodiester backbone. Representative ligands include either the four main naturally occurring DNA bases (i.e., thymine, cytosine, adenine or guanine) or other naturally occurring nucleobases (e.g., inosine, uracil, 5-methylcytosine or thiouracil) or artificial bases (e.g., bromothymine, azaadenines or azaguanines, etc.) attached to a peptide backbone through a suitable linker. PNAs are able to bind complementary ssDNA and RNA strands. Methods for making and using PNAs are disclosed in U.S. Pat. No. 5,539,082. Another type of modification that can be used to make oligonucleotides having the sequences described herein involves the use of non-nucleotide linkers (e.g., see U.S. Pat. No. 6,031, 091) between nucleotides in the nucleic acid chain which do not interfere with hybridization or optionally elongation of a primer. Yet other analogs include those that increase the binding affinity of the primer to a target nucleic acid and/or increase the rate of binding of the primer to the target nucleic acid relative to a primer without the analog.

All of the oligonucleotides of the present invention may be labeled for allowing the detection of amplification products. Essentially any labeling and detection system that can be used for monitoring specific nucleic acid hybridization can be used in conjunction with a labeled probe is desired. Included among the collection of useful labels are: radiolabels, enzymes, haptens, linked oligonucleotides, colorimetric, fluorometric, e.g., 6-carboxyfluorescein (FAM), carboxytetramethylrhodamine (TAMRA), or VIC (Applied Biosystems), or chemiluminescent molecules, and redox-active moieties that are amenable to electrochemical detection methods. In one embodiment, probes are labeled at one end with a reporter dye and with a quencher at the other end, e.g., reporters including FAM, 6-tetrachlorofluorescein (TED, MAX (Synthegen), Cy5 (Synthegen), 6-carboxy-X-rhodamine or 5 (6) -carboxy-X-rhodamine (ROX), and TAMRA and quenchers including TAMRA, BHQ (Biosearch Technologies) and QSY (Molecular Probes). Standard isotopic labels that can be used to produce labeled oligonucleotides include 3H, 355, 3zP, I2sl, sCo and 14C. When using radiolabeled probes, hybrids can be detected by autoradiography, scintillation counting or gamma counting.

Alternative procedures for detecting particular amplification products can be carried out using either labeled probes or unlabeled probes. For example, hybridization assay methods that do not rely on the use of a labeled probe are disclosed in U.S. Pat. No. 5,945,286 which describes immobilization of unlabeled oligonucleotide probe analogs made of peptide PNAs, and detectably labeled intercalating molecules which can bind double-stranded PNA probe/target nucleic acid duplexes. In these procedures, as well as in certain electrochemical detection procedures, such as those disclosed in PCT/US98/12082, PCT/US98/12430 and PCT/US97/20014, the oligonucleotide probe is not required to harbor a detectable label.

Detection of Vancomycin-Resistant Enterococci

In the hospital laboratory, routine culture for the detection of vancomycin-resistant Enterococcus from stool or anal swabs using selective media is a reliable method but may require up to 4-7 days for identification. Culture methods are also time consuming and expensive for laboratories performing a large number of specimens. For recovery of vancomycin-resistant enterococci in the laboratory, a selective medium containing vancomycin at a concentration of 6 μg/ml in agar is used. This medium also contains bile esculin, which is hydrolyzed to impart a black-brown color to Enterococcus colonies. Identification of suspect colonies and antimicrobial susceptibility tests are performed on Enterococcus spp., which also can take several days to perform.

The invention provides methods for detecting the presence or absence of one or more vancomycin-resistant enterococci in a biological sample from an individual. The methods include performing at least one cycling step that includes amplifying and detecting the amplification product. An amplification step includes contacting the biological sample with one or more oligonucleotide primers pairs to produce one or more amplification products if nucleic acid molecules from vancomycin-resistant enterococci present in the sample. Each of the target specific primer pairs generates an amplification product corresponding to the respective target nucleic acid, and, more importantly, each amplification product is different in size from the others. In this way, the targets can be identified based on the observed size of the amplification products. According to the invention, the method further includes detecting the presence or absence of PCR products using any method well known to those of ordinary skill in the art, as for example using labeled primers, fluorescent probes, etc. Multiple cycling steps can be performed, preferably in a thermocycler.

As used herein, “amplifying” refers to the process of synthesizing nucleic acid molecules that are complementary to one or both strands of a template nucleic acid (eg. vanA or vanB nucleic acid molecules). Amplifying a nucleic acid molecule typically includes denaturing the template nucleic acid, annealing primers to the template nucleic acid at a temperature that is below the melting temperature of each primer, and enzymatically elongating from each primer to generate an amplification product that consists of that flanked by the primer pair. The denaturing, annealing and elongating steps each can be performed once. Generally, however, the denaturing, annealing and elongating steps are performed several times such that the amount of amplification product is multiplied (often exponentially or doubled on each cycle, although exponential amplification is not required by the present methods disclosed here). Amplification typically requires the presence of deoxyribonucleoside triphosphates, a DNA polymerase enzyme (eg. PLATINUM® TAQ) and an appropriate buffer and/or co-factors for optimal activity of the polymerase enzyme (eg. MgCl 2 and/or KCl).

Since E. faecium and E. faecalis are the most predominant VRE, usually the method described in this invention will produce two amplification products (one from E. faecium or E. faecalis and the other from a vancomycin resistance gene (vanA, vanB, vanC1, vanC2/C3). Inadequate specimen collection, transportation delays, inappropriate transportation conditions or use of certain collection swabs (eg. calcium alginate or aluminum shaft) are all conditions that can affect the success and/or accuracy of the test results. Using the methods disclosed herein, gel-based detection of PCR products obtained after 35-40 cycling steps is indicative of one or more VRE present in the sample.

Representative biological samples that can be used in practicing the methods of the invention include anal or perirectal swabs, stool samples, blood, or body fluids. Biological sample collection and storage methods are known to those of skill in the art. Biological samples can be processed (eg. by standard nucleic acid extraction methods and/or using commercial kits) to release nucleic acid encoding vancomycin-resistance or, in some cases, the biological sample is contacted directly with the PCR reaction components and the appropriate oligonucleotides.

Within each thermocycler run, control samples can be cycled as well. Control nucleic acid can be amplified from a positive control sample using, for example, control primers. Positive control samples can also be used to amplify, for example, a plasmid construct containing a VRE nucleic acid molecule. Such a plasmid control can be amplified internally (eg. within each biological sample) or in separate samples run side-by-side with the patients' samples. Each thermocycler run should also include a negative control that, for example, lacks VRE template nucleic acid. Such controls are indicators of the success or failure of the amplification reaction. Therefore, control reactions can readily determine, for example, the ability of primers to anneal with sequence-specificity and to initiate elongation.

In one embodiment, the methods of the invention include steps to avoid contamination. For example, an enzymatic method utilizing uracil-DNA glycosylase is described in U.S. Pat. Nos. 5,035,996, 5,683,896 and 5,945,313 to reduce or eliminate contamination between one thermocycler run and the next. In addition, standard laboratory containment practices and procedures are desirable when performing methods of the invention. Containment practices and procedures include, but are not limited to, separate work areas for different steps of a method, containment hoods, barrier filter pipette tips and dedicated air displacement pipettes. Consistent containment practices and procedures by personnel are desirable for accuracy in a diagnostic laboratory handling clinical samples.

Conventional PCR methods or Real Time PCR can be used to practice the methods of the invention. Addition of selected fluorescent dyes to the reaction components allows the PCR to be monitored in real-time. An amplification product can be detected using a nucleic acid binding dye such as a fluorescent DNA binding dye (eg. SYBRGreen® or SYBRGold®, Molecular Probes). Upon interaction with the double-stranded nucleic acid, such nucleic acid binding dyes emit a fluorescence signal after excitation with light at a suitable wavelength. A nucleic acid binding dye such as a nucleic acid intercalating dye also can be used. When nucleic acid binding dyes are used, a melting curve analysis is usually performed for confirmation of the presence of the amplification product. Alternatively other formats that can be used for detection of amplification products which include, but are by no means limited to: individual tubes each with a different probe or comprising a plurality of probes; the wells of a 96-well or other multi-well microtiter plate; and a solid support such as a dipstick or a “DNA chip” where polynucleotide probes are immobilized to the support at different addresses in a spaced-apart configuration.

It is understood that the present invention is not limited by the configuration of one or more commercially available instruments.

Kits of the Invention

The invention further provides for kits to detect VRE. A test kit may contain one or more oligonucleotides of the invention. For example, the kit may contain one or more primers specific for one or more antibiotic resistance genes, the vanA or vanB gene, and optionally for particular species of bacterium as well as control primers. The kit is provided in the form of test components and, if present, the probe may be unlabelled or labelled. Preferably, if more than one labelled probe is present, each one is labelled with a different label.

In one embodiment, the kit will also optionally include test reagents necessary to perform the amplification reaction (eg. a polymerase, dNTPs, one or more salts, and/or a buffer, and/or reagents necessary to perform the hybridization reaction), reagents for pre-hybridization, hybridization, washing steps and/or hybrid detection. The kit may include as well standard samples to be used as negative and positive controls for each test.

In another embodiment, a test kit includes all reagents and controls to perform DNA amplification assays. Diagnostic kits are adapted for amplification by PCR (or other amplification methods) performed directly either from clinical specimens or from a bacterial colony. Components required for detection of antibiotic resistance genes and bacterial identification may be included.

It is understood that the use of the oligonucleotide primers described in this invention for bacterial detection and identification is not limited to clinical microbiology applications. In fact, these tests could be used by industries for quality control of food, water, pharmaceutical products or other products requiring microbiological control. These tests could also be applied to detect and identify bacteria in biological samples from organisms other than humans (eg. other primates, mammals, farm animals and live stocks). These diagnostic tools could also be useful for research purposes including clinical trials and epidemiological studies.

The invention will be further described in the following examples, which do not limit the scope of the invention described in the claims.

### EXAMPLES

### Example 1

### Oligonucleotide Primers

Oligonucleotide primers pairs directed to specific nucleotide sequences of E. faecium, E. faecalis, vanA, vanB, vanC1 and vanC2/3 were designed (Table 1). The E. faecium amplification product was 261 by in length, the E. faecalis amplification product was 473 by in length, the vanA amplification product was 373 by in length, the vanB amplification product was 139 by in length, the vanC1 amplification product was 82 by in length, and the vanC2/C3 amplification product was 676 by in length.

TABLE 1
Sequences of primers provided by this invention.

| | | SEQ |
|---|---|---|
| | | ID |
| Target | Sequence | NO: |
| E. faecium | 5′-TGC AAA ATG CTT TAG CAA CAG | 1 |
| | CC-3′ | |
| E. faecium | 5′-TCG TGT AAG CTA ACT TCG CGT | 2 |
| | AC-3′ | |
| E. faecalis | 5′-CGT ATT CTT GCG CTT GAT GAG | 3 |
| | C-3′ | |
| E. faecalis | 5′-GGG TGT CTT AGC TAG CGT TAA | 4 |
| | CG-3′ | |
| vanA | 5′-CGC GGA CGA ATT GGA CTA C-3′ | 5 |
| vanA | 5′-GGG CAG AGT ATT GAC TTC GTT | 6 |
| | C-3′ | |
| vanB | 5′-GGA AGC TAT GCA AGA AGC CAT | 7 |
| | G-3′ | |
| vanB | 5′-GGG AAA GCC ACA TCA ATA | 8 |
| | CGC-3′ | |
| vanC1 | 5′-GCT CCA ATC TGC ATT AAC GAC | 9 |
| | TG-3′ | |
| vanC1 | 5′-GCC AAT TTC AAT ACC CGC TAT | 10 |
| | CG-3′ | |
| vanC2/C3 | 5′-CGC AAT CGA AGC ACT CCA | 11 |
| | ATC-3′ | |
| vanC2/C3 | 5′-AAA GCC GTC TAC TAA TGA AAT | 12 |
| | GGC-3′ | |

All primers were synthesized on a 0.025 μm scale by Sigma-Aldrich (USA).

### Example 2

PCR Conditions.

For the PCR assay, a 1 μl aliquot of extracted nucleic acid was added to 14 μl of Master Mix (Table 2) in each PCR tube. A no-target control received 14 μl of Master Mix with 1 μl water.

TABLE 2
Master Mix

| Ingredient | Stock | Final concentration | μl |
|---|---|---|---|
| MgCl2 | 50 mM | 2.200 mM | 0.660 |
| dNTP | 10 mM | 0.300 mM | 0.450 |
| PCR buffer 10× | 10× | 1.000× | 1.500 |
| primers E. faecium | 10 μM | 0.200 μM | 0.300 |
| primers E. faecalis | 10 μM | 0.400 μM | 0.600 |
| primers vanA | 10 μM | 0.150 μM | 0.225 |
| primers vanB | 10 μM | 0.150 μM | 0.225 |
| primers vanC1 | 10 μM | 0.150 μM | 0.225 |
| primers vanC2/C3 | 10 μM | 0.150 μM | 0.225 |
| Platinum taq | 5 U/μL | 0.057 U/μL | 0.171 |
| H2O | — | — | 9.419 |
| | | Total: | 14 |

The PCR protocol is shown in Table 3.

TABLE 3
PCR Protocol

| No. of cycles | Temperature | Time | |
|---|---|---|---|
| 1 | 95° C. | 5 | min |
| 40 | 94° C. | 25 | sec |
| | 62° C. | 25 | sec |
| | 72° C. | 40 | sec |
| 1 | 72° C. | 3 | min |

### Example 3

Limit of Detection and Analytical Specificity.

For the following experiments, amplification was performed as described above in Examples 1 and 2.

Limit of Detection

The limit of detection was determined by using dilutions of different clinical isolates. The limit of detection claimed for the assay is 50 CFU/assay for E. faecium and 12 CFU/assay for E. faecalis.

Analytical Specificity

Control experiments were performed to determine if the primers described herein for detecting vancomycin-resistant enterococci cross-reacted with DNA from similar organisms or from organisms commonly found in the biological samples. For the cross-reactivity panels, the presence of microorganism DNA was initially confirmed by amplification of 16S rRNA. A total of 26 different culture isolates were evaluated included bacteria and yeast. All organisms were tested at 1.0×10⁶ CFU/assay. The list of organisms tested is shown in Table 4.

TABLE 4
Microorganisms tested in the analytical specificity study.

| Organism | Organism |
|---|---|
| Campylobacter coli | methicillin-resistant S. aureus |
| Campylobacter jejuni | methicillin-susceptible S. aureus |
| Candida albicans | Proteus mirabilis |
| Candida glabrata | Providencia alcalifaciens |
| Candida tropicalis | Pseudomonas aeruginosa |
| Enterococcus avium | Saccharomyces cerevisiae |
| Enterococcus hirae | Salmonella enteritidis serovar. Choleraesius |
| Enterococcus rafinosus | Salmonella enteritidis serovar. Enteritidis |
| Escherichia coli | Salmonella enteritidis serovar. Typhimurium |
| Haemophilus influenza | Serratia liquefaciens |
| Klebsiella pneumonia | Staphylococcus xylosus |
| Listeria innocua | Staphylococcus epidermidis |
| Listeria monocytogenes | Streptococcus pyogenes |

The primers described herein did not cross-react with any of the above-indicated isolates tested.

### Example 4.

Clinical Validation.

For the following experiments, amplification was performed as described above in Examples 1 and 2.

For the clinical evaluation, rectal swabs were collected from November 2007 to April 2008 from 187 patients at high risk for VRE colonization. Eligible participants included patients with five or more days in intensive care or hemato-oncology units. A single rectal rayon swab (Copan Diagnostics, USA) per patient was obtained by nursing staff using the liquid Stuart's BBL CultureSwab collection and transport system (BD diagnostics, USA).

Identification of VRE by Enterococcosel Plate Culture.

Each swab was used to inoculate a plate of Enterococcosel agar (BD diagnostics, USA) containing vancomycin at 6 μg/ml and then stored at −20° C. until DNA extraction for the PCR-based testing was performed. The cultures were then incubated aerobically at 35° C. for 3 days and positive colonies were analyzed using a GPI card in a VITEK equipment (Biomerieux, France). The Enteroccocus positive isolates were then studied for vancomycin resistance using E-test and the phenotypical approach to vanA or vanB genotypes using the teicoplanin disc susceptibility test. CSLI 2008 breakpoints were used for susceptibility interpretation.

Identification of VRE by the mtx-PCR Assay.

DNA was extracted from the swab with the DNExtract Swab Kit (TAAG Diagnostics, Chile) according to manufacturer instructions. The extracted DNA was kept at 4° C. until the PCR reaction was performed. PCR was performed as described above in Examples 1 and 2. This test is a multiplex PCR assay that simultaneously amplifies specific target sequences of Enterococcus faecium and Enterococcus faecalis species, in addition to vanA, vanB, vanC1 and vanC2/C3 genes. Additionally, a positive internal control was added to monitor if amplification inhibition occurs. Each of the target specific primer pairs generates an amplification product corresponding to the respective target nucleic acid, and, more importantly, each amplification product is different in size from the others, therefore based on the amplification product size can be inferred the target detected. Amplification products were analyzed by 2% agarose gel electrophoresis. PCR-negative controls for each bundle of samples processed at the same time were also included, and consisted of sterile water added to the last sample tube.

Data Analysis.

The results obtained with the PCR-based assay were first compared to the results obtained with Enterococcosel plate culture. Therefore, in this initial analysis, the Enterococcosel plate culture was considered as the gold standard. Following a recommended practice in the assessment of new diagnostic tests (Alonzo T., et al 1999. Statist. Med. 18:2987-3003), and because of the known low sensitivity of the Enterococcosel plate culture test, in a second round of analysis, the gold standard was switched to a composite reference standard that consisted of Enterococcosel plate culture and PCR followed by DNA sequencing of PCR products. In this composite reference standard analysis, positive VRE specimens were defined as those that tested positive by either one of the methods that constitute the composite scheme, otherwise the specimen is defined as a negative (ie. a sample is defined as positive when culture test resulted positive or, if culture test resulted negative and PCR resulted positive, when the sequence analysis of the PCR product confirmed that the sample had at least two targets: vanA/vanB and E. faecium/E. faecalis).

Statistical Analysis.

Confidence intervals for sensitivity, specificity, and positive and negative predictive values were based on exact binomial probabilities.

Evaluation of PCR Assay Versus Culture.

A total of 187 rectal swabs were evaluated. Using bacteria culture analysis, 125/187 (66.8%) samples were negative for any VRE and 62/187 (33.2%) samples were VRE positive. Using the PCR-based assay, 97/187 (51.9%) were VRE negative and 90/187 (48.1%) were VRE positive. Assuming bacteria culture method as the gold standard, the sensitivity and specificity values for the PCR-based assay were 96.8% (60/62) and 76.0% (95/125), respectively. The corresponding positive and negative predictive values in these samples were 67.7% and 97.9%, respectively. The performance of PCR compared to culture is shown in Table 5.

TABLE 5
Performance characteristics of PCR compared to culture

| | | Culture | | |
|---|---|---|---|---|
| | | Positive | Negative | Total |
| PCR | Positive | 60 | 30 | 90 |
| | Negative | 2 | 95 | 97 |
| | Total | 62 | 125 | 187 |

Evaluation of PCR and culture versus the composite reference standard. However, when the composite reference standard scheme is adopted as the gold standard in the analysis, 89 specimens were VRE positive and 98 VRE negative. The sensitivity and specificity values of the PCR-based assay were 97.8% (87/89) and 96.9% (95/98), respectively. The positive and negative predictive values in these samples were 96.7% and 97.9%, respectively. In this new scenario, with the composite reference standard as the gold standard, the sensitivity and specificity values of the Enterococcosel plate culture test were 69.7% and 100.0%, respectively. The positive and negative predictive values of this method in these samples were 85.5% and 100.0%, respectively. The difference in sensitivity between the molecular assay and culture was statistically significant (P<0.001). The performance of culture and PCR assay compared to the composite gold standard is shown in Table 6.

TABLE 6
Performance characteristics of culture and PCR compared to
composite gold standard

| | Sensitivity | | | Specificity | | |
|---|---|---|---|---|---|---|
| Method | No. | % | 95% CI | No. | % | 95% CI |
| Culture | 62/89 | 69.7 | 58.9-78.7 | 98/98 | 100 | 95.3-100 |
| PCR | 87/89 | 97.8 | 91.4-99.6 | 95/98 | 96.9 | 90.7-99.2 |

The PCR assay detected more positive specimens than did culture, and is therefore more sensitive than culture. A high rate of false negative results from rectal swab culture has been previously confirmed in the literature (D'Agata et al. 2002. Clin. Infect. Dis. 34:167-172).

1. A method to detect simultaneously nucleic acids from at least two or all of the following targets: E. faecium, E. faecalis, vanA, vanB, vanC1 and vanC2/C3 in a sample, comprising: a) contacting a biological sample suspected of comprising E. faecium, E. faecalis, vanA, vanB, vanC1 or vanC2JC3 nucleic acid with at least one E. faecium-specific oligonucleotide primer under conditions effective to amplify E. faecium nucleic acid and/or with at least one E. faecalis-specific oligonucleotide primer under conditions effective to amplify E. faecalis nucleic acid and/or with at least one vanA-specific oligonucleotide primer under conditions effective to amplify vanA nucleic acid and/or with at least one vanB-specific oligonucleotide primer under conditions effective to amplify vanB nucleic acid and/or with at least one vanC 1-specific oligonucleotide primer under conditions effective to amplify vanC1 nucleic acid and/or with at least one vanC2/C3-specific oligonucleotide primer under conditions effective to amplify vanC2/C3 nucleic acid, wherein the E. faecium-specific oligonucleotide primer comprises sequences which include sequences substantially corresponding to nucleotides 593 to 615 of the ddl gene from E. faecium, the complement thereof, or a portion thereof or sequences substantially corresponding to nucleotides 831 to 853 of the ddl gene from E. faecium, the complement thereof, or a portion thereof; and wherein the E. faecalis-specific oligonucleotide primer comprises sequences which include sequences substantially corresponding to nucleotides 213 to 234 of the ddl gene from E. faecalis, the complement thereof, or a portion thereof or sequences substantially corresponding to nucleotides 663 to 685 of the ddl gene from E. faecalis, the complement thereof, or a portion thereof; and wherein the vanA-specific oligonucleotide primer comprises sequences which include sequences substantially corresponding to nucleotides 558 to 576 of the vanA gene, the complement thereof, or a portion thereof or sequences substantially corresponding to nucleotides 909 to 930 of the vanA gene, the vanB-specific oligonucleotide primer comprises sequences which include sequences substantially corresponding to nucleotides 146 to 167 of the vanB gene, the complement thereof, or a portion thereof or sequences substantially corresponding to nucleotides 264 to 284 of the vanB gene, the complement thereof, or a portion thereof; and wherein the vanC1-specific oligonucleotide primer comprises sequences which include sequences substantially corresponding to nucleotides 248 to 270 of the vanC1 gene, the complement thereof, or a portion thereof or sequences substantially corresponding to nucleotides 307 to 329 of the vanC1 gene, the complement thereof, or a portion thereof; and wherein the vanC2/C3-specific oligonucleotide primer comprises sequences which include sequences substantially corresponding to nucleotides 100 to 120 of the vanC2/C3 gene, the complement thereof, or a portion thereof or sequences substantially corresponding to nucleotides 752 to 775 of the vanC2/C3 gene, the complement thereof, or a portion thereof; and b) detecting or determining the presence or amount of amplified nucleic acid.

2. The method of claim 1 wherein one of said targets is E. faecium which is detected with at least one E. faecium-specific oligonucleotide primer under conditions effective to amplify E. faecium nucleic acid, wherein the E. faecium-specific oligonucleotide primer comprises sequences which include sequences substantially corresponding to nucleotides 593 to 615 of the ddl gene from E. faecium, the complement thereof, or a portion thereof or sequences substantially corresponding to nucleotides 831 to 853 of the ddl gene from E. faecium, the complement thereof, or a portion thereof.

3. The method of claim 1 wherein one of said targets is E. faecalis which is detected with at least one E. faecalis-specific oligonucleotide primer under conditions effective to amplify E. faecalis nucleic acid, wherein the E. faecalis-specific oligonucleotide primer comprises sequences which include sequences substantially corresponding to nucleotides 213 to 234 of the ddl gene from E. faecalis, the complement thereof, or a portion thereof or sequences substantially corresponding to nucleotides 663 to 685 of the ddl gene from E. faecalis, the complement thereof, or a portion thereof.

4. The method of claim 1 wherein one of said targets is vanA which is detected with at least one vanA-specific oligonucleotide primer under conditions effective to amplify vanA nucleic acid, wherein the vanA-specific oligonucleotide primer comprises sequences which include sequences substantially corresponding to nucleotides 558 to 576 of the vanA gene, the complement thereof, or a portion thereof or sequences substantially corresponding to nucleotides 909 to 930 of the vanA gene, the complement thereof, or a portion thereof.

5. The method of claim 1 wherein one of said targets is vanB which is detected with at least one vanB-specific oligonucleotide primer under conditions effective to amplify vanB nucleic acid, wherein the vanB-specific oligonucleotide primer comprises sequences which include sequences substantially corresponding to nucleotides 146 to 167 of the vanB gene, the complement thereof, or a portion thereof or sequences substantially corresponding to nucleotides 264 to 284 of the vanB gene, the complement thereof, or a portion thereof.

6. The method of claim 1 wherein one of said targets is vanC1 which is detected with at least one vanC 1-specific oligonucleotide primer under conditions effective to amplify vanC1 nucleic acid, wherein the vanC1-specific oligonucleotide primer comprises sequences which include sequences substantially corresponding to nucleotides 248 to 270 of the vanC1 gene, the complement thereof, or a portion thereof or sequences substantially corresponding to nucleotides 307 to 329 of the vanC1 gene, the complement thereof, or a portion thereof.

7. The method of claim 1 wherein one of said targets is vanC2/C3 which is detected with at least one vanC2/C3-specific oligonucleotide primer under conditions effective to amplify vanC2/C3 nucleic acid, wherein the vanC2/C3-specific oligonucleotide primer comprises sequences which include sequences substantially corresponding to nucleotides 100 to 120 of the vanC2/C3 gene, the complement thereof, or a portion thereof or sequences substantially corresponding to nucleotides 752 to 775 of the vanC2/C3 gene, the complement thereof, or a portion thereof.

8. An oligonucleotide composition comprising a first oligonucleotide comprising sequences selected from the group consisting of i) sequences substantially corresponding to nucleotides 593 to 615 of the ddl gene from E. faecium, the complement thereof, or a portion thereof or sequences substantially corresponding to nucleotides 831 to 853 of the ddl gene from E. faecium, the complement thereof, or a portion thereof, wherein the oligonucleotide hybridizes to ddl gene from E. faecium; ii) sequences substantially corresponding to nucleotides 213 to 234 of the ddl gene from E. faecalis, the complement thereof, or a portion thereof or sequences substantially corresponding to nucleotides 663 to 685 of the ddl gene from E. faecalis, the complement thereof, or a portion thereof, wherein the oligonucleotide hybridizes to ddl gene from E. faecalis; iii) sequences substantially corresponding to nucleotides 558 to 576 of the vanAgene, the complement thereof, or a portion thereof or sequences substantially corresponding to nucleotides 909 to 930 of the vanA gene, the complement thereof, or a portion thereof, wherein the oligonucleotide hybridizes to vanA gene; iv) sequences substantially corresponding to nucleotides 146 to 167 of the vanB gene, the complement thereof or a portion thereof or sequences substantially corresponding to nucleotides 264 to 284 of the vanB gene, the complement thereof, or a portion thereof, wherein the oligonucleotide hybridizes to vanB gene; v) sequences substantially corresponding to nucleotides 248 to 270 of the vanC1 gene, the complement thereof, or a portion thereof or sequences substantially corresponding to nucleotides 307 to 329 of the vanC1 gene, the complement thereof, or a portion thereof, wherein the oligonucleotide hybridizes to vanC1 gene; and vi) sequences substantially corresponding to nucleotides 100 to 120 of the vanC2/C3 gene, the complement thereof or a portion thereof or sequences substantially corresponding to nucleotides 752 to 775 of the vanC2/C3 gene, the complement thereof, or a portion thereof, wherein the oligonucleotide hybridizes to vanC2/C3 gene.

9. The oligonucleotide composition of claim 8 wherein at least one oligonucleotide has the length and sequence of any of SEQ ID NOs: 1-2.

10. (canceled)

11. The oligonucleotide composition of claim 8 wherein at least one oligonucleotide has the length and sequence of any of SEQ ID NOs: 3-4.

12. (canceled)

13. The oligonucleotide composition of claim 8 wherein at least one oligonucleotide has the length and sequence of any of SEQ ID NOs: 5-6.

14. (canceled)

15. The oligonucleotide composition of claim 8 wherein at least one oligonucleotide has the length and sequence of any of SEQ ID NOs: 7-8.

16. (canceled)

17. The oligonucleotide composition of claim 8 wherein at least one oligonucleotide has the length and sequence of any of SEQ ID NOs: 9-10.

18. (canceled)

19. The oligonucleotide composition of claim 8 wherein at least one oligonucleotide has the length and sequence of any of SEQ ID NOs: 11-12.

20. A kit comprising oligonucleotides specific for detect vanA and vanB genes in a test sample, comprising oligonucleotides including sequences that substantially correspond to nucleotides 558 to 576 of the vanA gene, the complement thereof, or a portion thereof and sequences substantially corresponding to nucleotides 909 to 930 of the vanA gene, the complement thereof, or a portion thereof, and oligonucleotides comprising sequences substantially corresponding to nucleotides 146 to 167 of the vanB gene, the complement thereof, or a portion thereof and sequences substantially corresponding to nucleotides 264 to 284 of the vanB gene, the complement thereof, or a portion thereof.

21. The kit of claim 20 further comprising oligonucleotides specific for detect E. faecium and E. faecalis in a test sample, comprising oligonucleotides including sequences that substantially of the vanC2/C3 gene, the complement thereof, or a portion thereof or correspond to nucleotides 593 to 615 of the ddl gene from E. faecium, the complement thereof, or a portion thereof and sequences substantially corresponding to nucleotides 831 to 853 of the ddl gene from E. faecium, the complement thereof, or a portion thereof and oligonucleotides comprising sequences substantially corresponding to nucleotides 213 to 234 of the ddl gene from E. faecalis, the complement thereof, or a portion thereof or sequences substantially corresponding to nucleotides 663 to 685 of the ddl gene from E. faecalis, the complement thereof, or a portion thereof.

22. The kit of claim 20, further comprising oligonucleotides specific for detect vanC1 and vanC2/C3 genes in the test sample, comprising oligonucleotides comprising sequences substantially corresponding to nucleotides 248 to 270 of the vanC1 gene, the complement thereof, or a portion thereof and sequences substantially corresponding to nucleotides 307 to 329 of the vanC1 gene, the complement thereof, or a portion thereof, and oligonucleotides comprising sequences substantially corresponding to nucleotides 100 to 120 of the vanC2/C3 gene, the complement thereof, or a portion thereof and sequences substantially corresponding to nucleotides 752 to 775 of the vanC2/C3 gene, the complement thereof, or a portion thereof.

23. The kit of claim 22 further comprising oligonucleotides specific for detect E. faecium and E. faecalis in a test sample, comprising oligonucleotides including sequences that substantially correspond to nucleotide. 593 to 615 of the ddl gene from E. faecium, the complement thereof, or a portion thereof and sequences substantially corresponding to nucleotides 831 to 853 of the ddl gene from E. faecium, the complement thereof, or a portion thereof; and oligonucleotides comprising sequences substantially corresponding to nucleotides 213 to 234 of the ddl gene from E. faecalis, the complement thereof, or a portion thereof or sequences substantially corresponding to nucleotides 663 to 685 of the ddl gene from E. faecalis, the complement thereof, or a portion thereof.

24. The kit of claim 20 wherein the detection or determination of the amplification products is done using probes or DNA melting analysis.

25. The kit of claim 20 wherein an internal control is used to monitor amplification inhibition.
